(19) United States
(12) Patent Application Publication
(10) Pub. No.: US 2025/0032317 A1
(43) Pub. Date: Jan. 30, 2025

(54) DEVICE TO CREATE REPRODUCIBLE FENESTRATIONS IN A NON-VALVED TUBE SHUNT

(71) Applicant: Ohio State Innovation Foundation, Columbus, OH (US)

(72) Inventors: Joshua Evans, Columbus, OH (US); Lauren Eichaker, Columbus, OH (US); Faleh Alzoubi, Columbus, OH (US); Jack Laird, Columbus, OH (US); Caleb Mallory, Columbus, OH (US); Mallory Stewart, Columbus, OH (US); Natalie Zachariah, Columbus, OH (US)

(21) Appl. No.: 18/777,701

(22) Filed: Jul. 19, 2024

Related U.S. Application Data
(60) Provisional application No. 63/515,617, filed on Jul. 26, 2023.

(51) Int. Cl. A61F 9/007

(52) U.S. Cl. CPC A61F 9/00781; A61F 2240/005

(57) ABSTRACT

A fenestration template device includes a main body extending along a longitudinal axis between a first end and a second end of the device. The device includes an outer surface extending from the first surface to the second surface. The device includes a central lumen defined by the main body along the longitudinal axis, the central lumen extending from a first opening at the first surface to a second opening at the second surface. The device includes a plurality of side lumens defined by a plurality of side openings in the outer surface and extending from the side openings, through the main body, and into the central lumen. The device is configured for accepting a tube of an ocular implant (e.g., a glaucoma drainage implant) in the central lumen. The device is further configured for accepting a needle or suturing device in the plurality of side lumens.

### CROSS-REFERENCE TO RELATED APPLICATIONS

This application claims priority to U.S. Provisional Patent Application No. 63/515,617, filed Jul. 26, 2023, which is incorporated herein by reference in its entirety.

### BACKGROUND

The present disclosure relates generally to glaucoma drainage devices (e.g., tube shunts) including the implantation of such devices. Generally, glaucoma drainage devices are designed to divert aqueous humor from the anterior chamber to, for example, an external reservoir. Such devices are particularly useful in controlling intraocular pressure (IOP), among other conditions.

One such glaucoma drainage device is a non-valved Baerveldt Shunt which comprises a plate and a tube. The Baerveldt Shunt may be implanted with the plate anchored adjacent to or between two rectus muscles. The tube may be laid across the cornea with a beveled edge opening towards the cornea, and the tube may extend into the anterior chamber. The distal end of the tube, adjacent to the plate, may be ligated or sutured closed until the plate has time to encapsulate within the anatomy. After a time (e.g., 6 weeks), the ligation sutures dissolve, opening the drainage tube. Until such time, fenestrations are created in the tube to provide fluid drainage immediately after surgery.

To ensure proper drainage of aqueous humor from the anterior chamber, one or more fenestrations (i.e., small openings) may be created in the sidewall of the tube. However, current implantation techniques rely on a particular surgeon's chosen technique. There is currently a lack of predictability of outflow characteristics of tube shunt fenestrations due to the lack of a method to standardize the creation of fenestrations, leaving the number and size of fenestrations up to the surgeon. Additionally, the ligation of the base of the tube can be difficult and time-consuming, which can lengthen surgical time. The varied operation, including the tools chosen, may result in frequent complications, variable outcomes, and repeated surgeries in glaucoma patients.

Therefore, there exists a need for more consistent fenestration creation in tube shunt devices.

### SUMMARY

Described herein are systems, methods, and devices for creating consistent fenestrations glaucoma drainage devices (e.g., a non-valved, Baerveldt Shunt; or the Ahmed ClearPath device from New World Medical) during surgical procedures aimed at alleviating symptoms of Glaucoma.

In one aspect, a fenestration template device is disclosed, the device including: a main body extending along a longitudinal axis between a first surface at a first end of the device and a second surface at a second end of the device; an outer surface extending from a first edge at the first surface to a second edge at the second surface; a central lumen defined by the main body along the longitudinal axis, the central lumen extending from a first opening at the first surface to a second opening at the second surface; and a plurality of side lumens defined by a plurality of side openings in the outer surface and extending from the plurality of side openings, through the main body, and into the central lumen, wherein the device is configured for accepting a tube of an ocular implant (e.g., a glaucoma drainage implant) in the central lumen, and wherein the device is further configured for accepting a needle or suturing device in the plurality of side lumens.

In some implementations, the ocular implant is a non-valved Baerveldt Shunt. In some implementations, the device is configured for producing at least one fenestration in the tube of the ocular implant.

In some implementations, the at least one fenestration includes a single-sided fenestration extending through one side of a sidewall of the tube. In some implementations, the at least one fenestration includes a double-sided fenestration extending through both sides of a sidewall of the tube.

In some implementations, the plurality of side openings is arranged in a consistent pattern in an axial direction along the outer surface. In some implementations, the plurality of side lumens is arranged in a consistent pattern, wherein each of the plurality of side lumens has a diameter the same as an adjacent one of the plurality of side lumens.

In some implementations, the central lumen is sized to accept the tube of the ocular implant, and the plurality of side lumens is sized to accept the needle or suturing device.

In some implementations, a device, the sizing and spacing of the elements of the device is based on an optimized flow rate of aqueous humor from the anterior chamber of an eye. In some implementations, the flow rate is about 2.5 μL/min. In some implementations, a device, wherein the device is cylindrical. In some implementations, the plurality of side openings are arranged adjacent to the first end of the main body. In some implementations, an inner diameter of the tube of the ocular implant is about 0.32 mm, an outer diameter is about 0.64 mm, and a length is about 32 mm. In some implementations, a diameter of the plurality of side openings is about 0.1 mm or based on a 7-0 suturing needle diameter.

In some implementations, a device, further including: a spring attenuation mechanism configured to depress towards the central lumen to fenestrate the tube. In some implementations, a device, further including: at least one small rod disposed at an end of the main body configured to aid in occlusion of the tube.

In some implementations, a device, wherein the main body includes a first body half hingably coupled to a second body half via a hinge, the first and second body halves being defined along a plane parallel to the longitudinal axis, wherein the device may be opened and closed via the hinge to expose the central lumen for placement of the tube of the ocular implant.

In another aspect, a method of producing consistent fenestrations in a tube of an ocular implant is disclosed, the method including: providing a fenestration template device having a central lumen defined by a main body and a plurality of side lumens extending from an outer surface of the main body to the central lumen; providing the ocular implant having a base and a drainage tube; inserting the drainage tube into the central lumen of the fenestration template device; ligating a proximal end of the drainage tube adjacent to the base; inserting a needle or suturing device into at least one of the side lumens; and producing, by the needle or suturing device, at least one fenestration in the drainage tube, wherein the at least one fenestration is configured for an optimized flow rate of aqueous humor from the anterior chamber of an eye.

In some implementations, producing the at least one fenestration includes producing two or more fenestrations in the drainage tube. In some implementations, producing the at least one fenestration includes producing a through hole extending through both sidewalls of the drainage tube. In some implementations, producing the at least one fenestration includes producing a hole extending through only one sidewall of the drainage tube. In some implementations, a method, further including: threading a suture through the at least one fenestration in the drainage tube.

In some implementations, a system of producing consistent fenestrations in a tube shunt as described herein.

In some implementations, a device for producing consistent fenestrations in a tube shunt as described herein.

In some implementations, a method of producing consistent fenestrations in a tube shunt as described herein.

### BRIEF DESCRIPTION OF THE DRAWINGS

FIG. 1A is a side view of one side of an example fenestration template device showing the side openings, according to one implementation.

FIG. 1B is a top or bottom view showing a different side of the example fenestration template device of FIG. 1A adjacent to a ruler for dimensional context.

FIG. 1C is a front view of the example fenestration template device of FIG. 1A adjacent to a ruler for dimensional context.

FIG. 2 is an image of a 3D printed prototype fenestration template device, according to one implementation.

FIGS. 3A-3D show another implementation of a fenestration template device. Specifically, FIG. 3A shows a detail view of the fenestration template device with internal elements visible, according to one implementation.

FIG. 3B shows a front view of the fenestration template device of FIG. 3A.

FIG. 3C shows a side view of one side of the fenestration template device of FIG. 3A with the slit openings visible.

FIG. 3D shows a top or bottom view showing a different side of the fenestration template device of FIG. 3A.

FIG. 4 shows an image of a prototype fenestration template device with the slit openings visible, according to one implementation.

FIG. 5 shows another implementation of a fenestration template device including a hinge in the open configuration.

FIG. 6 depicts a data collection setup and experimental design setup, according to one implementation.

FIG. 7 shows a graph of changing fenestrations vs flow rate, according to one implementation.

FIG. 8 shows a graph depicting mass flow changing with time, according to one implementation.

FIG. 9 shows a graph of fenestrations depicting potential value, according to one implementation.

FIG. 10 depicts fluid flow at pseudo study state for one fenestration, wherein the top panel is the fluid flow, and the bottom panel is a pressure contour map.

FIG. 11 depicts fluid flow at pseudo study state for two fenestrations, wherein the top image is the fluid flow, and the bottom panel is a pressure contour map.

FIG. 12 depicts fluid flow at pseudo study state for one fenestration and shows the pressure gradient as throughout the fluid, according to one implementation.

FIG. 13 depicts fluid flow at pseudo study state for two fenestrations and shows the pressure gradient as throughout the fluid, according to one implementation.

### DETAILED DESCRIPTION

Described herein are systems, methods, and devices for creating consistent fenestrations glaucoma drainage devices (e.g., a non-valved, Baerveldt Shunt; or the Ahmed ClearPath device from New World Medical) during surgical procedures aimed at alleviating symptoms of Glaucoma. For example, in some implementations, a fenestration template device is disclosed, the device including: a main body extending along a longitudinal axis between a first surface at a first end of the device and a second surface at a second end of the device; an outer surface extending from a first edge at the first surface to a second edge at the second surface; a central lumen defined by the main body along the longitudinal axis, the central lumen extending from a first opening at the first surface to a second opening at the second surface; and a plurality of side lumens defined by a plurality of side openings in the outer surface and extending from the plurality of side openings, through the main body, and into the central lumen, wherein the device is configured for accepting a tube of an ocular implant (e.g., a glaucoma drainage implant) in the central lumen, and wherein the device is further configured for accepting a needle or suturing device in the plurality of side lumens.

In another implementation, a method of producing consistent fenestrations in a tube of an ocular implant is disclosed, the method including: providing a fenestration template device having a central lumen defined by a main body and a plurality of side lumens extending from an outer surface of the main body to the central lumen; providing the ocular implant having a base and a drainage tube; inserting the drainage tube into the central lumen of the fenestration template device; ligating a proximal end of the drainage tube adjacent to the base; inserting a needle or suturing device into at least one of the side lumens; and producing, by the needle or suturing device, at least one fenestration in the drainage tube, wherein the at least one fenestration is configured for an optimized flow rate of aqueous humor from the anterior chamber of an eye.

While the systems, methods, and devices of this disclosure are generic to a variety of glaucoma drainage devices and shunts, a specific description of the non-valved Baerveldt shunt is provided for ease of reference. Thus, the subject of this disclosure is not limited to any particular manufacturer, shape, or style of ocular implant, and the systems, methods, and devices disclosed herein may be utilized with a variety of ocular implants.

An example glaucoma drainage device and shunt is a medical device used to treat glaucoma, a condition that can lead to increased intraocular pressure (IOP) within the eye, potentially causing damage to the optic nerve and leading to vision loss if left untreated. One example device includes a small, flexible tube (e.g., a shunt) made of biocompatible materials, such as silicone or polypropylene, and a flat, plate-like reservoir that sits on the surface of the eye. The implantation of the example device is performed in a surgical procedure by an ophthalmologist. During the surgery, the tube is inserted into the anterior chamber, and the plate is positioned under the conjunctiva, a thin membrane covering the white part of the eye.

The example device and the associated shunt or tube diverts the aqueous humor, the fluid that nourishes the eye and maintains its shape, from the anterior chamber of the eye to the plate reservoir. By doing so, excess fluids drain away from the eye, reducing the intraocular pressure (IOP). To control the flow of aqueous humor and regulate the amount of drainage, the surgeon creates fenestrations or small holes in the tube. These fenestrations are strategically placed along the length of the tube. The number and size of the fenestrations may vary depending on the specific needs of the patient and the severity of their glaucoma. By having fenestrations, the surgeon can control the rate of fluid drainage, preventing excessive drainage and potential complications like hypotony (very low intraocular pressure).

The example device is then covered by the surrounding tissues, and the incisions are closed. After the surgery, the device continues to facilitate drainage of the aqueous humor, regulating intraocular pressure to prevent damage to the optic nerve and preserve vision.

There is currently a lack of predictability of outflow characteristics of tube shunt fenestrations due to the lack of a method to standardize the creation of fenestrations, leaving the number and size of fenestrations up to the surgeon. The exact spacing, size, and positioning of the fenestrations may be inconsistent or inadequate, resulting in frequent complications, variable outcomes, and repeated surgeries in glaucoma patients. Additionally, the ligation of the base of the tube can be difficult and time-consuming, which can lengthen surgical time.

Towards improving non-valved tube shunt procedures, this disclosure describes a novel device to standardize the creation of fenestrations in tube shunts (e.g., at the surgical site or at the bedside). The device will standardize the size and placement of the fenestrations by acting as a guide to place a blade or needle. In this way, the guide will allow for predetermined fenestrations to be placed at standard depths.

### Example Devices

Described herein is an example device that serves as a guide to allow surgeons to create standardized and repeatable fenestrations in the tube portion of the implant (e.g., the tube of a non-valved, Baerveldt shunt). This allows the surgery process to occur in a shorter amount of time and creates more predictable outcomes which will benefit both surgeons and patients.

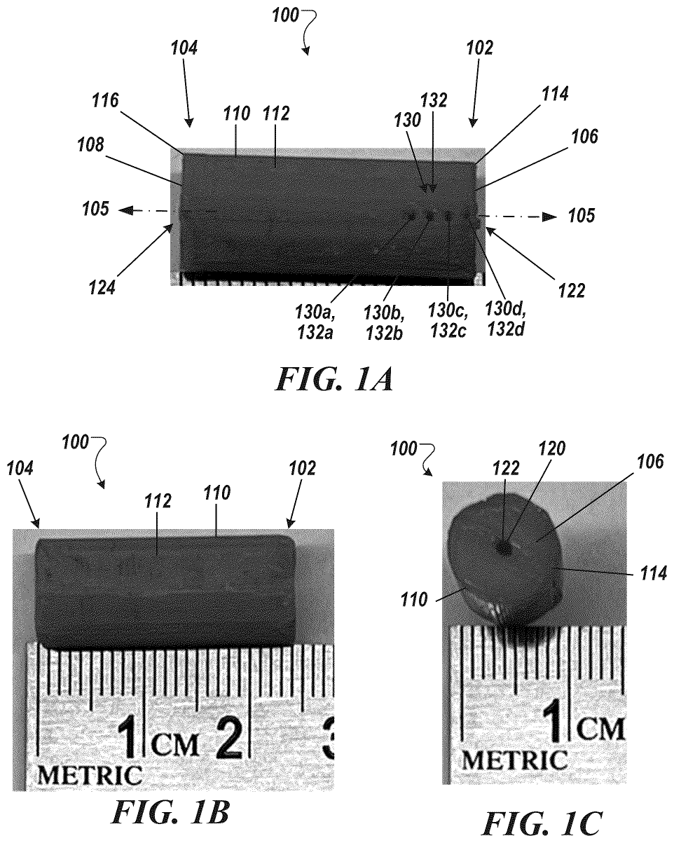

FIGS. 1A-5 show different views of example fenestration template devices. However, the example devices of FIGS. 1A-5 represent particular embodiments of the disclosure, and this disclosure is not limited by any one embodiment. For example, the size of the claimed device is not limited by an individual example. For example, in other implementations, the device may be substantially smaller depending on the ophthalmic surgical procedure.

FIGS. 1A-1C show a fenestration template device 100 according to one implementation. FIG. 1A shows a first side view of the device 100, FIG. 1B shows a top or bottom view (e.g., 90 degrees offset from the view in FIG. 1A) of the device 100, and FIG. 1C shows a front view of the device 100. FIGS. 1B and 1C include a reference measurement device to provide dimensional context. However, any dimensions or measurements presented in the figures are exemplary only and are not meant to limit the scope of the disclosure.

The device 100 includes a main body 110 extending along a longitudinal axis 105. The main body 110 extends between a first surface 106 on a first end 102 of the main body 110 and a second surface 108 on a second end 104 of the main body 110. An outer surface 112 extends from the first surface 106 and the second surface 108 to define a first edge 114 and a second edge 116 respectively therebetween.

The main body 110 is substantially cylindrical in shape. However, in other implementations, the main body of the device may be a different shape (e.g., a rectangular, hexagonal, or octagonal prism).

A central lumen 120 is defined by the main body 110 along the longitudinal axis 105. The central lumen 120 extends from a first opening 122 defined at the first surface 106 and a second opening 124 defined at the second surface 108. The central lumen 120 is substantially cylindrical in shape, and is configured for accepting a tube of an ocular implant (e.g., the flexible tube of any of the glaucoma drainage implants described herein). The dimensions and geometry of the central lumen 120 substantially match that of tube of an ocular implant. For example, the diameter of the central lumen 120 may be in the range of 0.1 mm-2.0 mm (e.g., between 0.3 mm and 0.8 mm, such as 0.64 mm). In some implementations, the tube of the ocular implant to be inserted into the central lumen has an outer diameter of about 0.64 mm and an inner diameter of about 0.32 mm. However, in other implementations, the tube of the ocular implant may have a different outer diameter substantially matching the central lumen and a different wall thickness.

A plurality of side openings 132 are defined by the outer surface 112. A plurality of side lumens 130 are defined by and extend from the plurality of side openings 132. Each of the plurality of side lumens 130 extend from the plurality of side openings 132, through the main body 110, and into the central lumen 120. Thus, the plurality of side openings 132 are in fluid communication with the central lumen 120 via the plurality of side lumens 130. The plurality of side openings 132 and the plurality of side lumens 130 are arranged adjacent to the first end 102 of the device 100. However, in other implementations, the side openings and corresponding lumens may be disposed in the middle of the device or near the second end of the device, or a combination of other arrangements.

As shown in FIG. 1A, the plurality of side openings 132 includes four side openings 132a, 132b, 132c, and 132d corresponding to four side lumens 130a-130d. However, in other implementations, a different number of side openings or lumens are present in the device (e.g., 1, 2, 3, 5, 6, 7, 8, 10, or more openings and corresponding lumens). The plurality of side openings 132 and the corresponding plurality of side lumens 130 are configured for accepting a needle or suturing device.

Each of the plurality of side openings 132 and the plurality of side lumens 130 have a diameter substantially the same as each other, which substantially matches that of a needle or suturing device. However, in other implementations, one or more of the side openings and corresponding side lumen may have a diameter different from another side opening and corresponding lumen. In some implementations, the diameter of one of the side openings and corresponding side lumen is between about 0.03 mm an 0.5 mm (e.g., about 0.1 mm or matching a #7-0 suture or suturing needle).

The plurality of side openings 132 and corresponding plurality of side lumens 130 are arranged in a linear and evenly-spaced pattern aligned with the longitudinal axis 105. However, in other implementations, the side openings are arranged in a different pattern (e.g., unevenly-spaced, non-linearly arranged, or disposed at a different angle relative to the central lumen). In other implementations, a second set of openings and corresponding side lumens are defined on an opposite side of the main body of the device. For example, in some implementations, a second set of side openings and side lumens may align with the plurality of side lumens 130 and plurality of side openings 132 on the first side of the main body 110. The sizing and spacing of the plurality of side lumens 130 and plurality of side openings 132 may be based on an optimized flow rate of aqueous humor from the anterior chamber or an eye (e.g., in the range of 1 to 5 μL/min, such as 2.5 μL/min).

The device 100 (and other implementations described herein) is configured for producing consistent fenestrations (e.g., four small holes axially aligned along the longitudinal axis of the device) in a tube of an ocular implant. For example, the device 100 is configured for producing consistent fenestrations in the drainage tube of a non-valved, Baerveldt shunt. Consistent fenestrations may be produced before, during, or after a surgical procedure. For example, the fenestrations may be produced before the base of the shunt is implanted or after the base of the shunt is implanted under the conjunctiva of an eye.

In use, the drainage tube is inserted into the central lumen 120 of the fenestration template device 100. A proximal end of the drainage tube may be ligated or sutured to the base of the ocular implant device. Then, a needle or suturing device is inserted into at least one of the side openings 132 and the corresponding side lumens 130 (e.g., a suturing device coupled to a suturing thread). The fenestrations are created in the drainage tube by inserting the needle or suturing device through the side lumen 130 and into the drainage tube (e.g., all the way through both sidewalls of the drainage tube, or through only one sidewall of the drainage tube). The consistently spaced side lumens 130 thus provide a template for inserting the needle or suturing device and producing the fenestrations (i.e., the drainage holes).

The size, number, and spacing of the fenestrations in the drainage tube are configured for an optimized flow rate of aqueous humor from the anterior chamber of an eye. For example, in a situation where more aqueous humor must be drained from the anterior chamber, a surgeon may take advantage of all the side openings 132 of the device 100 (e.g., all four of the openings 132a-132d and corresponding side lumens 130a-130d of the device 100). In other implementations where less aqueous humor must be drained from the anterior chamber, a surgeon may only use a portion of the side openings 132a-132d of the device 100 (e.g., only one of the openings 132a and the corresponding side lumen 130a of the device 100).

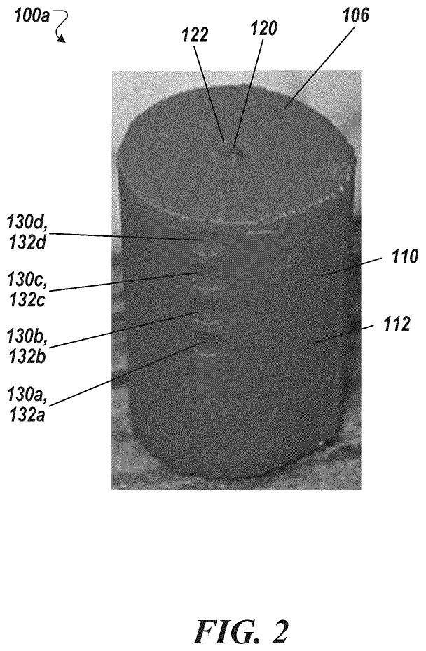

FIG. 2 shows a prototype fenestration template device 100a that is substantially similar to the device 100 in FIGS. 1A-1C. The device 100a is a 3D printed implementation used in experimental testing described further below. However, in some implementations, the device disclosed herein and used in practice may be 3D printed.

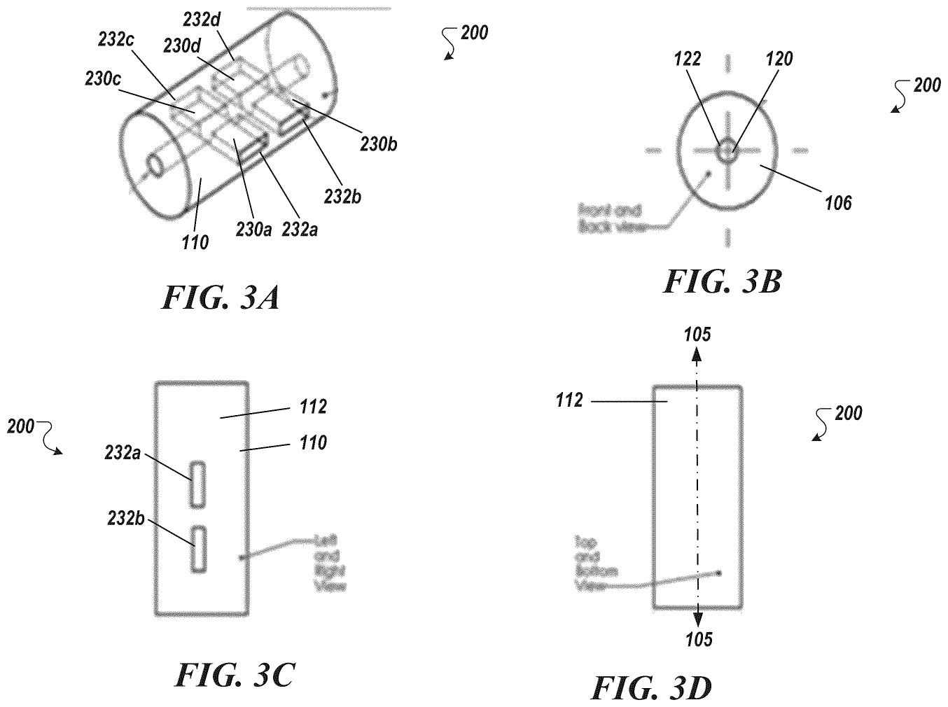

FIGS. 3A-3D show another implementation of the fenestration template device of the present disclosure. The device 200 shown in FIGS. 3A-3D is substantially similar to the device 100 of FIGS. 1A-1C except as described below.

The device 200 includes a plurality of side openings 232a-232d and corresponding side lumens 230a-23d that extend from the outer surface 112 to the central lumen 120. However, the device 200 includes two side openings 232a, 232b on one side of the main body 110 and two side openings 232c, 232d on the opposite side of the main body 110 (e.g., on the opposite side of the longitudinal axis 105). Thus, the corresponding side lumens 230a, 230b are directly opposite from the side lumens 230c, 230d.

Furthermore, the plurality of side openings 232a-232d are wider than the plurality of side openings 132 of the device 100. The plurality of side openings 232a-232d and corresponding side lumens 230a-230d are rectangular in shape (e.g., a longitudinal slit).

When the device 200 is used to create fenestrations in the drainage tube of an ocular implant, the fenestrations in the drainage tube may include a longitudinal slit or an oblong shape. In such implementations, the oblong shape may provide for increased drainage of aqueous humor when needed.

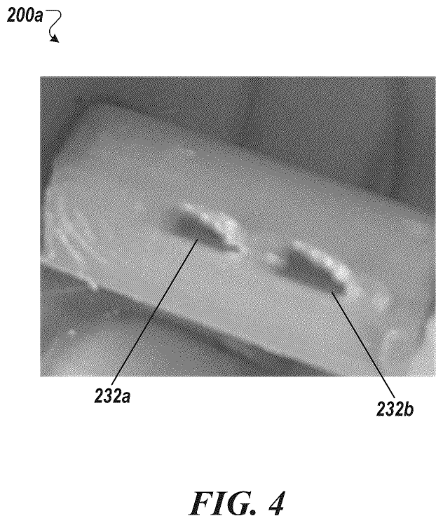

FIG. 4 shows an image of a prototype fenestration template device 200a as shown in FIGS. 3A-3D. The device 200a is a 3D printed implementation used in experimental testing described further below. However, in some implementations, the device disclosed herein and used in practice may be 3D printed.

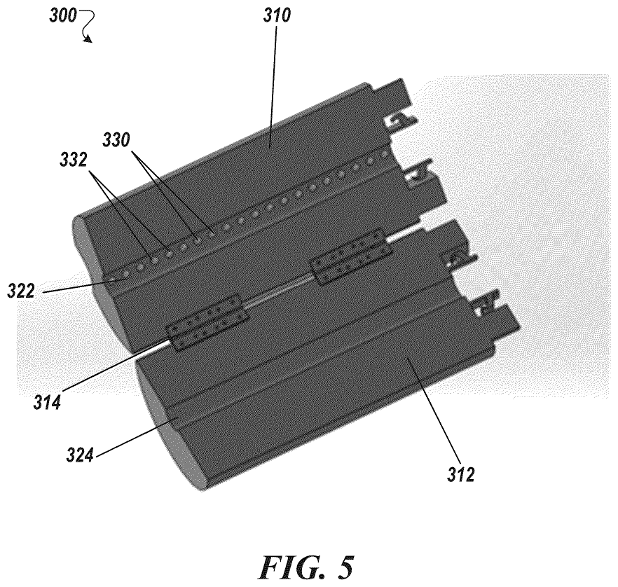

FIG. 5 shows a fenestration template device 300 according to another implementation. The device 300 is configured to clamp onto a tube (e.g., the shunt of an ocular implant), and may be used with a standardized blade for consistent fenestrations. The device 300 is similar to the device 100 of FIGS. 1A-1C except as described below.

The device 300 includes a first body half 310 and a second body half 312 hingably coupled to the first body half 310. The first body half 310 and the half 312 are coupled via a hinge 314. The first body half 310 and the half 312 are defined along a plane parallel to the longitudinal axis 105. The device 300 may be opened and closed via the hinge 314 (i.e., moved between an open configuration and a closed configuration), wherein the open configuration is shown in FIG. 5. In the open configuration, the central lumen is exposed for placement of the tube of the ocular implant.

The central lumen is defined by the first lumen half 322 on the first body half 310 and the second lumen half 324 on the half 312. The first lumen half 322 is in fluid communication with the plurality of side openings 332 and the corresponding side lumens 330. As shown, the device 300 includes side lumens 330 along the entire longitudinal length of the device.

The device 300 may include a spring attenuation mechanism configured to be pressed down towards the central lumen to fenestrate the tube therein. In some implementations, the central lumen may include a small rod on one or both ends of the central lumen to aid in occlusion of the tube.

Compared to existing methods, the devices shown and described in FIGS. 1A-5 provide a controllable and consistent system for producing holes or fenestrations in a tube of an ocular implant. Rather than the subjective fenestration creation of current systems, the example devices provide a consistent spacing between the fenestrations, leading to a more controllable and consistent outflow of aqueous humor. The intraocular pressure can thus be controlled more closely compared to current systems.

### Experimental Study and Results

A study was conducted to investigate a novel device for the standardized intraoperative preparation of non-valved glaucoma tube shunts.

### Introduction

Glaucoma refers to a category of eye conditions that involve damage to the optic nerve and is characterized by high intraocular pressure (IOP). This optic nerve damage causes pain and discomfort; in chronic cases, it can lead to long-term vision loss and sometimes blindness. After the failure of preliminary treatment such as drops and medication, late-stage glaucoma patients often undergo surgical intervention in which a drainage shunt is implanted to lower IOP. The shunt tube implantation surgery remains variable in both procedures and outcomes as there does not exist a one-size-fits-all method or tool for implant and procedure customization.

There is currently a lack of predictability of outflow characteristics of tube shunt fenestrations due to the lack of a method to standardize the creation of fenestrations, leaving the number and size of fenestrations up to the surgeon. Additionally, the ligation of the base of the tube can be difficult and time-consuming, which can lengthen surgical time.

Towards improving non-valved tube shunt procedures, this study describes a novel device to standardize the creation of fenestrations in tube shunts at the bedside. The device standardizes the size and placement of the fenestrations by acting as a guide to place a blade or needle. In this way, the guide will allow for predetermined fenestrations to be placed at standard depths. Additionally, a preliminary mathematical model has been created to predict the outflow characteristics of the novel device as a function of fenestration number. Validated mathematical models hold clinical value in predicting outflow characteristics and tailoring patient care.

Glaucoma tube shunt surgical procedures are being utilized more commonly in practice over recent decades after the movement away from trabeculectomies. The surgical procedure itself consists of four steps. Step one involves the insertion of the shunt plate (with a tube attached) to the supratemporal sclera. Step two involves ligation of the tube with a dissolvable suture at the base of the plate. Step three involves trimming the tube and insertion through a scleral-corneal tunnel into the anterior chamber of the eye. Step four, for a non-valved tube, involves the creation of fenestrations in the tube using a needle of variable size. Needle choice is up to the surgeon. In current practice, the tube is ligated at the end of the tube to prevent excess drainage of aqueous humor from the eye immediately following surgery before healing and scar formation. This suture dissolves after approximately six weeks and the main tube is unobstructed. At this point, a capsule has formed during healing that prevents excessive fluid loss. During these first 6 weeks, fenestrations are created in the tube between the eye and the ligated end to allow for immediate IOP reduction prior to suture dissolution. Patients who have failed trabeculectomy or have secondary glaucoma with high filtration failure turn to tube shunt surgical procedures to aid in reducing Intraocular Pressure or IOP.

Standard practice to reduce risk for Glaucoma today is reducing IOP, with non-invasive efforts, yet these methods often lack in reaching targeted IOP levels. Aqueous shunts (glaucoma drainage devices) help filter the aqueous humor out from the eye and into the subconjunctival space, via a silicone tube. This silicone tube further drains the aqueous humor from the anterior chamber to a plate, acting as a void, located on the sclera of the eye (usually within the supertemporal quadrant). These drainage devices currently offer inconsistent results; this may be due to patient-to-patient variability and/or implant variability. One study found ocular cell death for patients was correlated with IOP's lower than 5 mmHg, higher than 22 mmHg, or <20% reduction from the preoperative IOP. While patient variability has not yet been standardized, it is known that Baerveldt (non-valved) tube shunt procedures are variable in nature.

Creating fenestrations in the tube portion of a non-valved (Baerveldt) tube shunt implant is a common surgical method used to eliminate high levels of IOP immediately after implantation. Non-valved tube shunts require temporary flow restriction through the silicone tube until encapsulation of the endplate occurs. “Tube fenestration is a technique in which venting slits are placed proximal to a tube ligature to allow egress of aqueous humor before tube opening”. Patients that received tube fenestrations in the early postoperative period had higher rates of hypotony and hypotony-related complications relative to patients that did not receive fenestrations. Complications associated with aqueous shunts are immediate hypotony, excessive capsule fibrosis, clinical failure, erosion of the tube or plate edge, strabismus, and rarely infection. Further, the tube shunt procedure requires the silicone tube to be inserted within the anterior chamber of the eye, long-term consequences can be damage to the corneal endothelium. Due to procedural variation between patients, there is a larger room for complication(s) with the physical shunt tube drainage system. In existing procedures, the size, shape, and number of fenestrations are up to the discretion of the treating physician.

By improving the consistency of non-valved tube shunt procedures surgeons may focus on data that is mostly patient-derived. One way this may be achieved is via standardizing the size, shape, and number of fenestrations when using non-valved tube shunts. Having data specific to each patient could allow surgeons to better evaluate the IOP and correlate patient preoperative pressures to the fenestration outlet pressure needed to alleviate eye pain. There is therefore a need for a way to guide surgeon-to-surgeon procedural replication when using non-valved Glaucoma shunt tubes. Micro perfusion flow experiments in monkeys and rabbits have shown that there is a capsule around the explant which provides the main resistance to aqueous humor outflow. The standard tube also provides no resistance to the outflowing aqueous humor through the tube shunt, with physiological perfusion flow rates at around 2 to 4 microliters per minute. Knowing this, further standardization can be focused on the fenestrations and suturing during the surgical procedure, and variation associated with the silicone tube can be assumed negligible.

Current surgical practice tends to involve correlating the choice of the number of fenestrations to the starting IOP of the patient. However, there is currently a paucity of mechanistic studies that explain the success and variability associated with utilizing this correlation. This study seeks to set the groundwork for investigating the correlation between the number of fenestrations in a non-valved tube shunt and tube shunt flow in vitro. This study also seeks to explore the creation of a novel device for standardizing the size, number, and spacing of fenestrations. In standardizing the practice associated with creating fenestrations in non-valved tube shunt implants, this study may provide a way to isolate the study of patient-related factors from procedure-related factors in tube shunt procedures to potentially improve patient outcomes and surgical repeatability.

### Materials and Methods

Two different models were created to study pseudo-steady-state fluid flow through a virtual representation of a tube with fenestrations. Flow rate models were created in both SolidWorks and COMSOL that quantified flow rate and pressure and experimental design was created to understand both models.

### 2.1 SolidWorks Model

The SolidWorks computational fluid dynamics model stems from an accurate 3D model of the shunt tube with an inlet, and outlet, as well as varying number of fenestrations. The dimensions of the tube were based on current market shunt tube specifications with an inner diameter of 0.32 mm, an outer diameter of 0.64 mm, and a length of 32 mm. The fenestration diameter was approximated to be 0.1 mm based on microscopic measurement of the 7-0 suturing needle diameter. The input flow rate was based on aqueous humor production of the eye being 2.5 μL/min. The outlet was “plugged” off to direct flow toward the fenestrations. The outputs were chosen based on the number of fenestrations being tested and resulted in outflowing flow rates from each fenestration. Multiple iterations were run until the steady state flow was realized. In addition, assumptions were made including steady state, one-directional laminar flow, with 0 pressure outside the tube.

### 2.2 COMSOL Model

The COMSOL model utilized COMSOL Multiphysics 6.0 (Build: 405) Design Modules, MEM Module, Multiphysics, Cad Import, and CFD modules. Different parameters were utilized, and geometry was pulled via Barnhart Tube sizing including an inner diameter of 0.32 mm. Density and viscosity were pulled from aqueous humor properties, and a resistor was added via a silicone flap with properties pulled from Silicone PDMS 1110. The COMSOL model consists of multiple rectangles based on the fenestration number. These rectangles are then merged via a union to create a microfluidic valve. This model was created by emulating the COMSOL non-valved microfluidic chamber and making modifications to inlet and outlet flow along with dimension properties. One limitation of this model is it does not factor in the distance traveled via a non-fenestrated tube and focuses on emulating the valve. As surgical procedure states surgeons cut the tube and due to this variability, a starting value was selected at 600 micrometers. In addition, assumptions were made including steady state, one-directional laminar flow, with 0 pressure outside the tube.

### 2.3 Experimental Design

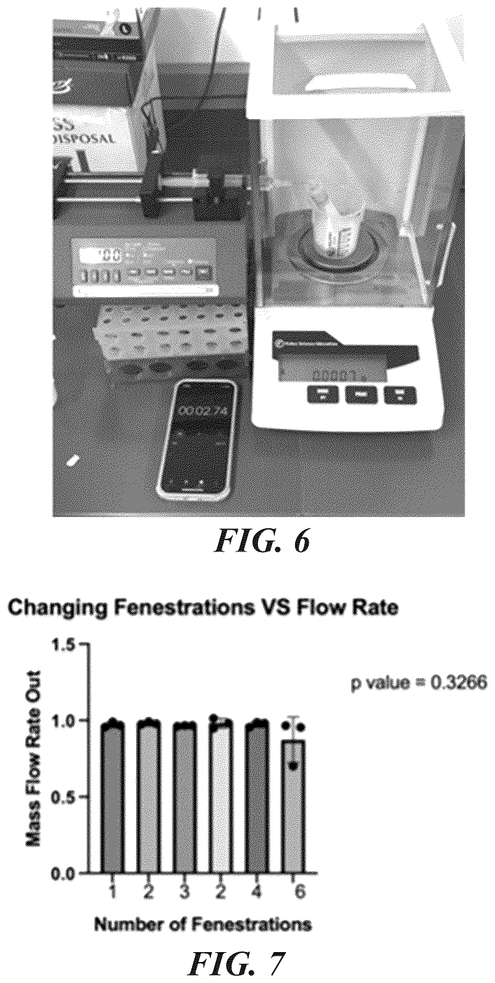

The two flow models were emulated using a fluid dynamic experimental design. The experimental test flow bench consisted of a New Era Pump Systems NE-1000 one-channel programmable syringe pump, a Beckton Dickinson 10 mL syringe, a 14-gauge Lock cannula, Fisher Science Education Compact scale, a collection beaker for outflowing water, and 18 fenestrated shunt tube samples, as shown in FIG. 6. Six types of fenestrations were used with three replicates each, which include one, two, and three single fenestrations and one, two, and three through fenestrations. This correlates to 2, 4, and 6 fenestrations on opposite sides of the tube. A new 7-0 suturing needle was used to create each fenestration in all of the tubes. Shunt tubes were then ligated downstream of the fenestrations to mimic clinical conditions and tap water at room temperature emulating aqueous humor was run through the occluded tube forcing liquid out of each fenestration. After confirmation that all fenestrations had fluid flow, and the tube was alluded samples were loaded onto a syringe cannula and placed on the syringe pump. The syringe pump was then turned on allowing fluid flow at a rate of 1 microliter per minute or 2.5 microliters per minute. Each hole variation was run for two minutes, 30 seconds to allow flow, 60 seconds of data collection, and the remaining 30 seconds for consistency. Each trial was recorded and mass data in grams was collected every second with a Fisher Science Education Compact scale. This data was then analyzed, and the results are discussed below.

Experimental conditions were analyzed using GraphPad Prism and consisted of a one-way ANOVA test to determine if fenestration number and fenestration geometry affect the flow rate of water (grams/second) through the tube (P<0.05).

### Results and Discussion

The results of this study incorporated both experimental design and multiple mathematical models.

### 3.1 Testing

First statistical testing was conducted on the experiment confirming the conservation of energy. The sum of the flow rates out must equal the flow rate initially put into the system. The test found that this proved to be true with no statistical difference between the fenestrations or a difference between single fenestrations and fenestrations through the tube. Statistical testing was conducted utilizing GraphPad Prism to compare hole flow rate and the p-value between the six variable groups was 0.3266 which is larger than the acceptable p-value of 0.05 as shown in FIG. 7. The graph in FIG. 7 depicts in flow rate does not vary with the number of fenestrations. A one-way ANOVA showed no significance at a p-value of 0.3266 confirming expected results for initial data of the experiment.

The sample with six fenestrations showed a higher variation with an outlier within the data set. This was likely caused due to a smaller fenestration, causing the liquid to get stuck in the tube and not land on the scale. As discussed in the materials and methods section practitioners utilize different cutting techniques, and even when the same practitioner uses the same microscope, and the same technique some variability between fenestrations may still occur.

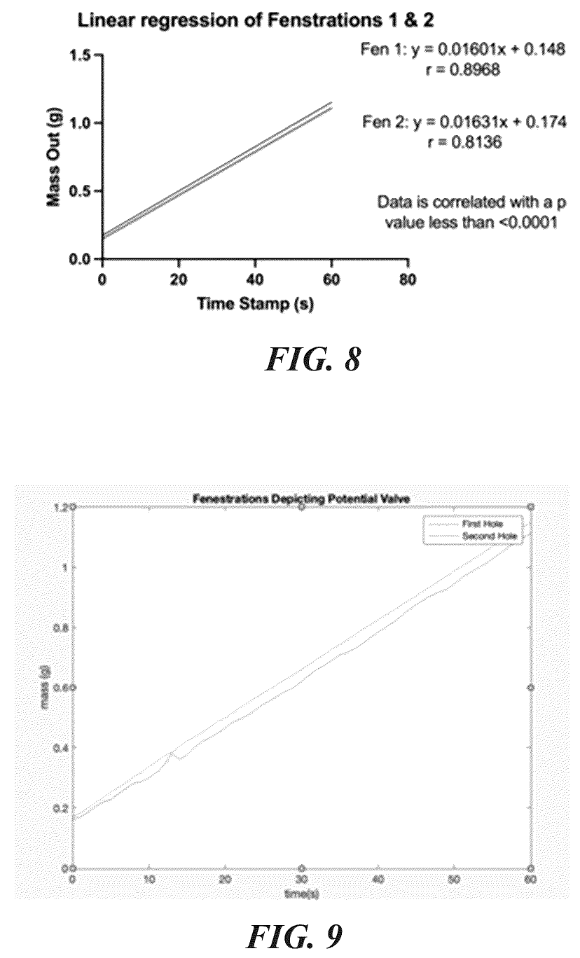

After confirming the conservation of energy and collecting data to compare to the preliminary mathematical model, time-stamped data was collected for each second and mass values, and a linear regression was performed utilizing GraphPad prism to confirm steady state. A comparative study between a single hole and a through hole showed no statistical difference from the mass collection. The results can be seen in FIG. 8 showing a high correlation between the two fenestration lines with a p-value of 0.0001 which is significantly smaller than the acceptable p-value of 0.05. While this result was hypothesized, the r values were lower than expected at r=0.8968, and 0.8136 so further investigation was done of the raw data on Matlab as depicted in FIG. 9. Specifically, FIG. 9 depicts raw data of each fenestration 1 and fenestration 2 from Matlab software. Three replicates were averaged, and the results are depicted.

Further analysis was conducted via Matlab and it was revealed that with one fenestration every five to ten seconds the mass collection would decrease and then increase again. It was further hypothesized that with the decreased flow rate of 1 mL/min to prevent slipping the pressure was not high enough during different time points to fully open the fenestration causing a valved effect. Further testing must be conducted to understand this phenomenon.

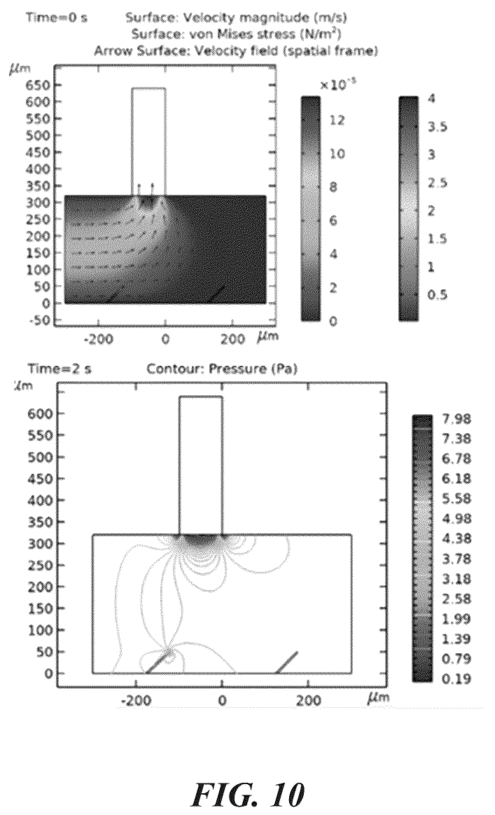

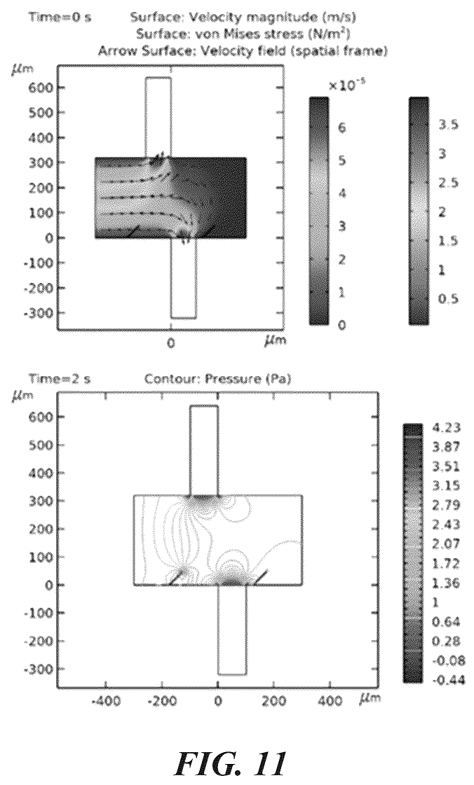

Additional investigation was conducted with a second model utilizing COMSOL, and a silicone flap to emulate a valve. One fenestration vs two fenestrations is depicted in FIG. 10, and FIG. 11 shows how fluid flows within the tube and the different pressures act at steady state. Two non-valved silicone-based resistors act as a potential emulation of a valve in future modeling. One current limitation of the model is the lack of a time-dependent study comparing the natural pressure rise within the eye, and the valve. This model aims to show a pseudo-steady state where aqueous humor production equals the flow rate out within the tube.

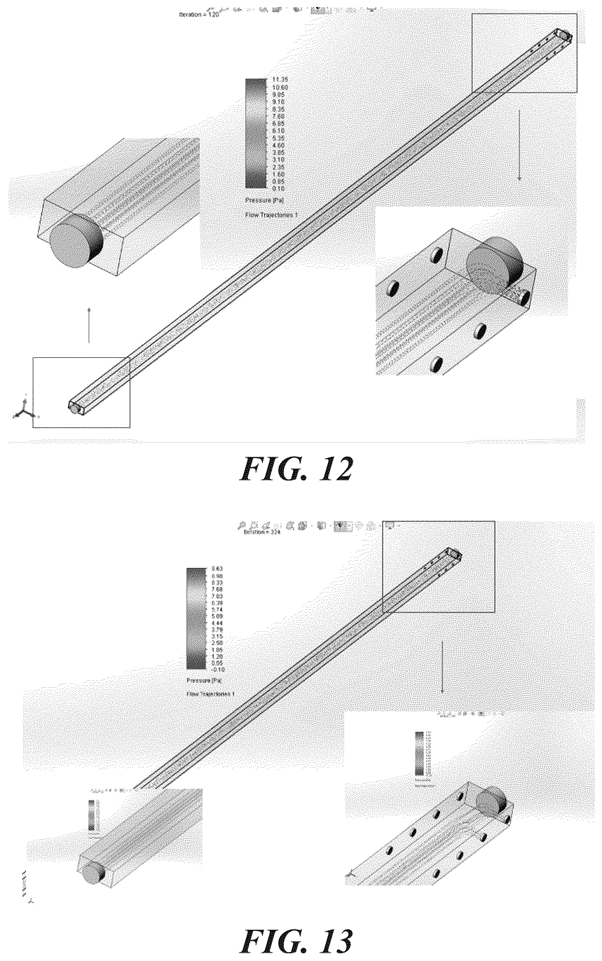

After creating a valved tube with the silicone flap, a nonvalue model was created to emulate an average higher flow rate of 2.5 mL/min with a higher number of fenestrations. SolidWorks was utilized to understand fluid flow properties, and these results are shown in FIGS. 12 and 13. FIG. 12 is the fluid flow within the tube and FIG. 13 depicts the results of a pressure study. The models demonstrate typical fluid dynamics laws and suggest that models that analyze changes in outflow resistance as a function of fenestration number may be a valuable next step.

### 3.2 Creating a Standardized Device

The computational models showed that the outflow through tube shunts follows traditional laws of fluid dynamics, but raises questions related to the effect of flow resistance and outflow velocity. In practice, when using a needle to create a fenestration, the shape of the needle creates a slit and not a hole; this provides for the possibility of valve-like behavior as the fenestrations respond to the IOP and the outflow of aqueous from the patient's eye. This motivates standardization of the placement and the size of the needle used to create fenestrations.

Therefore, a SolidWorks model was utilized to create a preliminary device design to assist surgeons with consistent fenestrations as shown in FIG. 5. The device aims to allow for consistent fenestrations by assisting in location placements, support to prevent depth variation, equal applied pressures to the tube during fenestration creation, and a suture holder to assist with occluding the tube. In addition, the device was purposely sized up so microscopes would no longer be necessary to make fenestrations, thereby reducing surgical time. Finally, attached needles and blades will be provided to allow for consistent insertion into the tube, towards minimizing microstructural variation.

The design in FIG. 5 utilizes a hinge to allow for proper placement onto the tube. Post placement, a spring attenuation mechanism would allow surgeons to press down and fenestrate the tube. A variation of holes will be placed on the side of the device allowing for different inserts to create fenestrations in the same place and location. In addition, two small rods will be placed at the end of the design to help surgeons occlude the tube. Location placement will be consistent with the blade's attenuation in each tube because of the consistent fenestration locations. Future studies must be conducted to understand blade design and its impacts.

To understand the feasibility of the device, design a 3D-printed device was created utilizing SolidWorks. It consists of a capsule-like design, with a 7.5-millimeter (about 0.3 in) device diameter. The inner hole where the shunt tube sits has a radius of 0.75 mm (about 0.03 in). The length of the device is 16 millimeters (about 0.63 in) and the slits to allow for surgical fenestrations of the tube are 2.45 mm (about 0.1 in) which was the same size of a 15-degree surgical blade which is currently used to make fenestrations in the operating room. The device's initial testing was printed using a photosensitive Creativity Resin 3D printer. The fabrication used UV light to crosslink the polymer and the device was processed using isopropyl alcohol to remove excess resin in-between the fenestration locations. These 3D printed models were not effective, due to the dried resin within the holes, and a second 3D model was printed using an extrusion printer to allow for more accurate hole sizes. In addition, the 3D models were printed and spliced laterally, where each layer was created horizontally as seen in FIGS. 3A-4.

By changing the reference frame of the 3D printers, the tolerance of the printer increased yielding better results. This method proved to be effective as shown in FIG. 2, with reference to FIGS. 1A-1C. FIGS. 1A-2 also show the challenges of 3D printing resin with variation between each layer allowing for potential fenestration variability. Challenges with this method also include size constraints and processing methods. In other implementations of the present disclosure, an injection moldering machine is used for tighter tolerances. In addition, material compatibility must be considered, adding a small 3D-printed device to the current shunt tubes on the market can allow for easier access to the necessary standardization for future projects. This standardization could allow for consistency in fluid flow out and potentially allow for a machine to predict the number of holes necessary to allow for the best outcome for the patient.

Some limitations of this experimental design were the mass of air—when working with such small mass values the weighing scale changes with movement, and with the device set up as seen in FIG. 6 the back door could not be closed due to the experiment set up causing potential error. To mitigate this error all testing was conducted in a large room with minimal airflow. An additional limitation existed in the experimental setup. The mass of the water flowing through the tube was not collected before 20-25 seconds due to a necessary initial pressure build-up before study state flow. Finally, an additional limitation is the steady state flow of the pump.

The purpose of this study is not to conclude whether more fenestrations will assist the patient as that requires additional study. The number of fenestrations required may correlates to a differential equation relating outflow resistance and IOP, as well as constants related to patient-related factors. The study showed that flow through the tube shunt follows traditional laws of fluid dynamics, and developed a standardizing non-valved tube shunt procedure by using a guide for creating more uniform fenestrations.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A fenestration template device comprising:
a main body extending along a longitudinal axis between a first surface at a first end of the device and a second surface at a second end of the device;
an outer surface extending from a first edge at the first surface to a second edge at the second surface;
a central lumen defined by the main body along the longitudinal axis, the central lumen extending from a first opening at the first surface to a second opening at the second surface; and
a plurality of side lumens defined by a plurality of side openings in the outer surface and extending from the plurality of side openings, through the main body, and into the central lumen,
wherein the device is configured for accepting a tube of an ocular implant in the central lumen, and
wherein the device is further configured for accepting a needle or suturing device in the plurality of side lumens.

2. The device of claim 1, wherein the device is configured for producing at least one fenestration in the tube of the ocular implant.

3. The device of claim 2, wherein the at least one fenestration includes a single-sided fenestration extending through one side of a sidewall of the tube.

4. The device of claim 2, wherein the at least one fenestration includes a double-sided fenestration extending through both sides of a sidewall of the tube.

5. The device of claim 1, wherein the plurality of side openings is arranged in a consistent pattern in an axial direction along the outer surface.

6. The device of claim 1, wherein the plurality of side lumens is arranged in a consistent pattern, wherein each of the plurality of side lumens has a diameter the same as an adjacent one of the plurality of side lumens.

7. The device of claim 1, wherein the central lumen is sized to accept the tube of the ocular implant, and wherein the plurality of side lumens is sized to accept the needle or suturing device.

8. The device of claim 1, wherein the sizing and spacing of the elements of the device is based on an optimized flow rate of aqueous humor from the anterior chamber of an eye.

9. The device of claim 8, wherein the flow rate is about 2.5 μL/min.

10. The device of claim 1, wherein the plurality of side openings are arranged adjacent to the first end of the main body.

11. The device of claim 1, wherein an inner diameter of the tube of the ocular implant is about 0.32 mm, an outer diameter is about 0.64 mm, and a length is about 32 mm.

12. The device of claim 1, wherein a diameter of the plurality of side openings is about 0.1 mm or based on a 7-0 suturing needle diameter.

13. The device of claim 1, further comprising:
a spring attenuation mechanism configured to depress towards the central lumen to fenestrate the tube.

14. The device of claim 1, further comprising:
at least one small rod disposed at an end of the main body configured to aid in occlusion of the tube.

15. The device of claim 1, wherein the main body comprises a first body half hingably coupled to a second body half via a hinge, the first and second body halves being defined along a plane parallel to the longitudinal axis,
wherein the device may be opened and closed via the hinge to expose the central lumen for placement of the tube of the ocular implant.

16. A method of producing consistent fenestrations in a tube of an ocular implant, the method comprising:
providing a fenestration template device having a central lumen defined by a main body and a plurality of side lumens extending from an outer surface of the main body to the central lumen;
providing the ocular implant having a base and a drainage tube;
inserting the drainage tube into the central lumen of the fenestration template device;
ligating a proximal end of the drainage tube adjacent to the base;
inserting a needle or suturing device into at least one of the side lumens; and
producing, by the needle or suturing device, at least one fenestration in the drainage tube, wherein the at least one fenestration is configured for an optimized flow rate of aqueous humor from the anterior chamber of an eye.

17. The method of claim 16, wherein producing the at least one fenestration includes producing two or more fenestrations in the drainage tube.

18. The method of claim 16, wherein producing the at least one fenestration includes producing a through hole extending through both sidewalls of the drainage tube.

19. The method of claim 16, wherein producing the at least one fenestration includes producing a hole extending through only one sidewall of the drainage tube.

20. The method of claim 16, further comprising:
threading a suture through the at least one fenestration in the drainage tube.
